(19) United States
(12) Patent Application Publication
(10) Pub. No.: US 2012/0277445 A1
(43) Pub. Date: Nov. 1, 2012

(54) METHOD FOR PRODUCING PROPYLENE OXIDE

(75) Inventors: Tomonori Kawabata, Toyonaka-shi (JP); Hirotsugu Kano, Niihama-shi (JP); Avelino Corma, Valencia (ES)

(73) Assignee: Sumitomo Chemical Company, Limited

(21) Appl. No.: 13/516,699

(22) PCT Filed: Dec. 6, 2010
(86) PCT No.: PCT/JP2010/072309

§ 371 Date: Jul. 18, 2012

(30) Foreign Application Priority Data
Dec. 18, 2009 (JP) 2009-287725

(51) Int. Cl. C07D 301/03; B01J 27/232; B01J 27/24; C07D 303/04; B01J 27/18

(52) U.S. Cl. 549/534; 502/208; 502/200; 502/174

(57) ABSTRACT

The present invention relates to a method for producing propylene oxide, comprising a step of bringing propylene, oxygen and a silver catalyst into contact with each other in the presence of water, wherein the silver catalyst is a catalyst prepared from (a) metallic silver, a silver compound or a mixture thereof, (b) phosphorus, a phosphorus-containing compound or a mixture thereof and (c) a carrier.

### TECHNICAL FIELD

The present invention relates to a silver catalyst for production of olefin oxide and a method for producing propylene oxide.

### BACKGROUND ART

A production method in which propylene is oxidized using oxygen has been known as a method for producing propylene oxide. For example, producing propylene oxide by reacting propylene with oxygen in the presence of water and a silver catalyst prepared by bringing silver powder and ((CH₃)₂N)₃PO into contact with each other is described in Japanese Examined Patent Application Publication No. S51-40051.

### SUMMARY OF INVENTION

The present invention provides methods for producing propylene oxide related to the following [1] to [18].

[1] A method for producing propylene oxide, comprising a step of bringing propylene, oxygen and a silver catalyst into contact with each other in the presence of water, wherein the silver catalyst is a catalyst prepared from (a) metallic silver, a silver compound or a mixture thereof, (b) phosphorus, a phosphorus-containing compound or a mixture thereof and (c) a carrier.
[2] The method according to [1], wherein the (c) comprises a metal carbonate excepting silver-containing metal carbonates, a metal oxide excepting silver-containing metal oxides or carbon as a main component.
[3] The method according to [1] or [2], wherein the (c) comprises an alkaline earth metal carbonate.
[4] The method according to any one of [1] to [3], wherein the amount of the (b) contained in the silver catalyst is in the range of 0.001 to 5 moles per mole of silver contained in the (a).
[5] The method according to any one of [1] to [4], wherein the (b) is a phosphorus-containing compound.
[6] The method according to any one of [1] to [5], wherein the silver catalyst is prepared by a method comprising:

a step of bringing the (a) and the (c) into contact with each other to obtain a composition thereof; and

a step of bringing the composition and the (b) into contact with each other.

[7] The method according to any one of [1] to [5], wherein the (a) is a silver compound or a mixture of metallic silver and a silver compound.
[8] The method according to [7], wherein the silver catalyst is prepared by a method comprising:

a step of bringing the (a) and the (c) into contact with each other to obtain a composition thereof;

a step of reducing the silver compound contained in the composition; and

a step of bringing the composition after reduction and the (b) into contact with each other.

[9] The method according to any one of [1] to [8], wherein the (a) is at least one silver compound selected from the group consisting of silver nitrate, silver carbonate and silver oxide.

[10] The method according to any one of [1] to [9], wherein the silver catalyst contains silver in an amount of at least 0.5% by mass based on the mass of the silver catalyst.

[11] The method according to any one of [1] to [10], wherein an amount of water is in the range of 0.2 to 10 moles per mole of propylene.
[12] A silver catalyst for production of olefin oxide which is a catalyst prepared from (a) metallic silver, a silver compound or a mixture thereof, (b) phosphorus, a phosphorus-containing compound or a mixture thereof and (c) a carrier.
[13] The silver catalyst according to [12], wherein the (a) is a silver compound, the silver catalyst being prepared by bringing the (a) and the (c) into contact with each other to obtain a composition thereof, reducing the silver compound contained in the composition, and then bringing the composition after reduction and the (b) into contact with each other.
[14] The silver catalyst according to [12] or [13], wherein the (c) comprises a metal carbonate excepting silver-containing metal carbonates, a metal oxide excepting silver-containing metal oxides or carbon as a main component.
[15] The silver catalyst according to any one of [12] to[14], wherein the silver compound is selected from the group consisting of silver nitrate, silver carbonate and silver oxide.
[16] The silver catalyst according to any one of [12] to [15], wherein the (c) comprises an alkaline earth metal carbonate.
[17] The silver catalyst according to any one of [12] to [16], wherein the (a) is at least one silver compound selected from the group consisting of silver nitrate, silver carbonate and silver oxide.
[18] The silver catalyst according to any one of [12] to [17], wherein the olefin oxide is propylene oxide.

### DESCRIPTION OF EMBODIMENTS

### <Silver Catalyst>

The silver catalyst according to the present invention is obtained from (a) metallic silver, a silver compound, or a mixture of these (also referred to briefly as “(a)” in the present specification), (b) phosphorus, a phosphorus-containing compound, or a mixture of these (also referred to briefly as “(b)” in the present specification) and (c) a carrier (also referred to briefly as “(c)” in the present specification).

This silver catalyst can be available for the production of olefin oxides such as propylene oxides.

The silver catalyst can be prepared, for example, by supporting (a) on (c) by bringing them into contact with each other and after this contact, mixing (b) with the composition of (a) and (c). In addition, it can also be obtained by mixing (b), (a) and (c) prior to this contact. The contact between (a) and (c) may be performed by mixing (a) and (c), for example.

When a silver compound or a mixture of metallic silver and a silver compound is used as (a), the silver catalyst is usually prepared through a step of reducing the silver compound. Although such a reduction may be performed prior to supporting (a) on (c), or may be performed after supporting (a) on (c), it is preferably performed prior to the contact of (a) and (b).

The silver catalyst generally has (b), (c) and metallic silver particles derived from (a), and has a structure in which the metallic silver particles derived from (a) are moderately dispersed in the silver catalyst and are supported on the carrier.

It is preferable that (a) is a silver compound. Examples of the preferred silver compounds include silver salts such as silver oxide, silver carbonate, silver nitrate, silver sulfate, silver cyanide, silver chloride, silver bromide, silver iodide, silver acetate, silver benzoate and silver lactate; and silver complexes such as silver acetylacetonate. Silver nitrate, silver carbonate, silver oxide or a mixture of at least two selected from these is preferable as the silver compound, and silver nitrate is more preferable.

The content of silver (silver atoms) in the silver catalyst is preferably at least 0.1% by mass, and is more preferably at least 0.5% by mass based on the mass of the silver catalyst. In addition, the content of silver (silver atoms) in the silver catalyst is preferably not more than 95% by mass based on the mass of the silver catalyst.

It is preferable to use each of (a), (b) and (c) in preparation of the silver catalyst such that the content of silver (silver atoms) in the silver catalyst is in the above-mentioned range. Herein, the content of silver is obtained by ICP emission analysis. However, the content of silver can be confirmed by employing XRF analysis.

A stable phosphorus such as red phosphorus is usually used as the phosphorus in (b).

Since the catalytic activity of the silver catalyst can be further raised, a phosphorus-containing compound is preferred as (b). The phosphorus-containing compound may be an inorganic phosphorus compound or may be an organic phosphorus compound.

As the phosphorus-containing compound, trialkylphosphines such as trimethylphosphine and triethylphosphine; triarylphosphines such as triphenylphosphine and tris(2-methoxyphenyl)phosphine; monodentate diphenylalkylphosphines such as diarylalkylphosphine such as diphenylmethylphosphine and diphenylethylphosphine; bidentate bis(diphenylalkyl)phosphines such as 1,2-diphenylphospinoethane and 1,4-bis(diphenylphoesphino)butane; trialkylphosphites such as trimethylphosphite, triethylphosphite and tributylphosphite; triarylphosphites such as triphenylphosphite; phosphorus trioxide, phosphorus tetroxide, phosphorus pentoxide, phosphoric acid, phosphorous acid, phosphoric acid salts, phosphorous acid salts, hypophosphorous acid, hypophosphorous acid salts, pyrophosphoric acid, pyrophosphoric acid salts, polyphosphoric acid, polyphosphoric acid salts, metaphosphoric acid, metaphosphoric acid salts and the like are preferable, and phosphoric acid, phosphorous acid, hypophosphorous acid and phosphoric acid salts are more preferable.

Examples of the phosphoric acid salt include ammonium dihydrogen phosphate, diammonium hydrogen phosphate and ammonium phosphate.

Only one phosphorus-containing compound may be used alone as (b), or two or more compounds such as phosphoric acid and a phosphoric acid salt may be mixed and used as (b).

In the preparation of the silver catalyst, for an amount of (b) used, the range of 0.005 to 10 moles (molecular basis) per mole of silver atoms (atom basis) is preferable, and the range of 0.01 to 1 mole (molecular basis) is more preferable.

The content of (b) in the silver catalyst thus obtained is preferably 0.001 to 5 moles (molecular basis) per mole of silver atoms (atom basis) in the silver catalyst, and more preferably 0.01 to 1 moles (molecular basis) per mole of silver atoms (atom basis) in the silver catalyst.

In the present specification, a carrier is a material that holds a catalyst material or disperses a catalyst material therein (Shouji Shida (editor); “Chemical Dictionary,” Morikita Publishing, p. 743, published Jan. 26, 1985), and refers to a material that holds or disperses (a).

As (c) carrier, one is usually selected that can support metallic silver particles and does not change in property in the preparation process of the silver catalyst. As such a carrier, a carrier containing a metal carbonate (except for silver-containing metal carbonates), a metal oxide (except for silver-containing metal oxides) or carbon as a main component is preferable. Herein, “a carrier containing a metal carbonate, metal oxide or carbon as a main component” is a concept including carriers consisted of a metal carbonate, metal oxide or carbon, carriers obtained by adhering particles of metal carbonates or metal oxides with a small amount of binder, and carriers in which a metal carbonate or metal oxide is molded with a molding agent or the like. Materials falling into either (a) or (b) simultaneously are not included in (c), even in the case the material falls into the definition of the above-mentioned carrier.

Preferable examples of the metal carbonate of (c) include alkaline earth metal carbonates and transition metal carbonates, and alkaline earth metal carbonates are preferable among these.

Examples of the alkaline earth metal carbonates include magnesium carbonate, calcium carbonate, strontium carbonate and barium carbonate. Among these, calcium carbonate, strontium carbonate and barium carbonate are preferable.

Alkaline earth metal carbonates having a specific surface area of 1 to 70 m²/g measured by nitrogen adsorption of the BET method are preferable.

The alkaline earth metal carbonate may be used as it is as the carrier, may be used as the carrier after fixing particles of the alkaline earth metal carbonate each other using a suitable binder, and the alkaline earth metal carbonate may be mixed with a molding agent and molded by extrusion molding, press molding or the like to use as the carrier. Among these, it is preferable to use the alkaline earth metal carbonate as it is as the carrier.

Examples of the metal in the metal oxide include metals other than silver, such as Ti, V, Mn, Mg, Ca, Sr, Ba, Fe, Co, Cd, Eu, Al, Mo, Ni, Zn, Cu, Ga, In, Sn and W.

A metal oxide having a crystal form of a rock salt structure, spinel-type structure, fluorite-type structure, wurtzite-type structure, rutile-type structure, bixbite-type structure, ilmenite-type structure, pseudobrookite-type structure or perovskite-type structure may be used as (c).

Examples of the metal oxides having a rock salt structure include TiO, VO, MnO, MgO, CaO, SrO, BaO, FeO, CoO, NiO, CdO and EuO.

Examples of the metal oxides having a spinel-type structure include MgAl₂O₄, SrAl₂O₄, CuAl₂O₄, MoAl₂O₄, MnAl₂O₄, FeAl₂O₄, CoAl₂O₄, NiAl₂O₄, ZnAl₂O₄, CdAl₂O₄, γ-Fe₂O₃, MgCo₂O₄, Co₃O₄, CuCo₂O₄, ZnCo₂O₄, MgV₂O₄, MnV₂O₄, FeV₂O₄, CoV₂O₄, ZnV₂O₄, CdV₂O₄, MgGa₂O₄, Mg1n₂O₄, SnMg₂O₄, TiMg₂O₄, VMg₂O₄, TiZn₂O₄, SnZn₂O₄ and WZn₂O₄.

Examples of the metal oxide having a fluorite-type structure include HfO₂, ZrO₂, CeO₂, ThO₂ and UO₂.

Examples of the metal oxide having a wurtzite-type structure include BeO and ZnO.

Examples of the metal oxide having a rutile-type structure include GeO₂, SnO₂, PbO₂, TiO₂, VO₂, NbO₂, TaO₂, CrO₂, MoO₂, WO₂, β-MnO₂, TcO₂, α-ReO₂, RuO₂, OsO₂, RhO₂, IrO₂ and PtO₂.

Examples of the metal oxide having a bixbite-type structure include β-Fe₂O₃.

Examples of the metal oxide having an ilmenite-type structure include FeTiO₃, NiVO₃ and MgVO₃.

Examples of the metal oxide having a pseudobrookite-type structure include FeTiO₅.

Examples of the metal oxide having a perovskite-type structure include MgTiO₃, CaTiO₃, SrTiO₃, BaTiO₃, CdTiO₃, PbTiO₃, CaZrO₃, SrZrO₃, BaZrO₃, PbZrO₃, SrHfO₃, BaHfO₃, SrMoO₃, BaMoO₃, CaCeO₃, SrCeO₃, PbCeO₃, YAlO₃, LaAlO₃, LaVO₃, YVO₃, LaCrO₃, LaMnO₃ and LaFeO₃.

Examples of the carbon usable in (c) include activated carbon, carbon black, graphite, carbon nanotubes and graphene, among which graphite and graphene are preferred.

The aforementioned metal carbonate, metal oxide and carbon are commercially available. Such a commercial product may also be used without alteration as (c), or may be used as (c) after purifying and molding the commercial product by a well-known method.

In the preparation of the silver catalyst, the amount of (c) used is not limited so long as the silver content in the silver catalyst is in the aforementioned range. The amount of (c) used is preferably 0.1 to 200 parts by mass per part by mass of silver, and more preferably 0.5 to 100 parts by mass per part by mass of silver.

The content of (c) in the silver catalyst thus obtained is preferably 5 to 99.9 parts by mass per part by mass of silver, and more preferably 10 to 70 parts by mass per part by mass of silver.

In the silver catalyst, it is preferable that (a) is silver nitrate, silver oxide or silver carbonate, (b) is a phosphoric acid salt, and (c) is an alkaline earth metal carbonate, and it is more preferable that (a) is silver nitrate, (b) is ammonium dihydrogen phosphate, diammonium hydrogen phosphate or ammonium phosphate, and (c) is strontium carbonate.

Although the silver catalyst may contain other metal atoms at a trace amount, it is preferable to make the incorporation of alkaline metals such as lithium, sodium, potassium, rubidium and cesium to be as little as possible in order for the catalytic action not to be significantly hindered. More specifically, it is preferable to select a silver compound so as to make the content of alkaline metal components in the silver catalyst to be 1500 or less ppm by mass based on the mass of the silver catalyst. The content of such an alkaline metal component in the silver catalyst can be obtained by ICP emission analysis or XRF analysis.

When (a) contains a silver compound, the silver compound is usually reduced during silver catalyst preparation. The reduction of the silver compound is preferably performed after the contact of (a) and (c). When (a) contains a silver compound, the silver compound may be contained in the silver catalyst thus obtained. However, the silver compound is preferably completely reduced during silver catalyst preparation.

Hereinafter, preparation of the silver catalyst will be explained specifically.

When the silver-containing composition described later is obtained by contacting (a) and (c), a method comprising Step I and Step II is given as the method for preparing the silver catalyst.

Step I: a step of bringing (a) and (c) into contact with each other to obtain a composition of these (in the present specification, it is referred to as “silver-containing composition”);

Step II: a step of bringing the silver-containing composition thus obtained in Step I and (b) into contact with each other

The method comprising Step I and Step II is usually performed when (a) includes metallic silver.

Step I can be performed in the same manner as Step 1 described later. Step II can be performed in the same manner as Step 3 described later.

The “silver-containing composition” is considered to be a morphology in which metallic silver particles are supported on a carrier.

When (a) is a silver compound or a mixture of metallic silver and a silver compound, a method comprising the following Step 1, Step 2 and Step 3 is given as the method for preparing the silver catalyst.

Step 1: a step of bringing a silver compound and a carrier into contact with each other to obtain a composition of these;

Step 2: a step of reducing the silver compound contained in the composition;

Step 3: a step of bringing the composition after reduction and (b) into contact with each other

The preparation method comprising Step 1, Step 2 and Step 3 is preferable because metallic silver fine particles can be obtained by reduction of the silver compound, and a silver catalyst in which the metallic silver fine particles are sufficiently dispersed in the carrier is obtained.

Hereinafter, a preparation method of a silver catalyst will be explained with a preparation method comprising Step 1, Step 2 and Step 3 as an example.

In Step 1, it is preferable to bring the silver compound and the carrier into contact with each other in the presence of a solvent. When a solvent is used, it is preferable to prepare a dispersion liquid of the carrier (hereinafter this dispersion is referred to as “carrier dispersion”) and a dispersion or solution of the silver compound (hereinafter this dispersion and solution are collectively referred to as “silver compound solution”) in advance, and mix the carrier dispersion and the silver compound solution. The solvent for the carrier dispersion preparation may be the same as or different from the solvent for the silver compound solution preparation.

Examples of the solvent used for the silver compound solution preparation in Step 1 include water; ethers such as tetrahydrofuran; hydrocarbons such as toluene and hexane; and mixed solvents of at least two kinds selected from these. The solvent for silver compound solution preparation is preferably one that easily dissolves the silver compound used, and water is particularly preferable among these.

The kind of solvent and the amount of that can be appropriately selected according to the kind of silver compound.

When the silver compound is dissolved in the solvent, after the silver compound and the solvent are mixed, the mixture may be heated or cooled as necessary, and the temperature at this time can be adjusted in the range of 0 to 200° C.

In addition, filtration or the like may be performed in order to remove a small amount of undissolved components remaining after dissolving.

An acid may be added to the solvent used for the silver compound solution preparation.

Acids that can be added to the solvent for the silver compound solution preparation may be either an inorganic acid or an organic acid.

Examples of the inorganic acid include hydrochloric acid, nitric acid, nitrous acid, sulfuric acid and perchloric acid.

Examples of the organic acid include aliphatic carboxylic acids such as acetic acid, oxalic acid, propionic acid, butyric acid, citric acid, maleic acid, fumaric acid and tartaric acid; and aromatic carboxylic acids such as benzoic acid, dicarboxybenzene, tricarboxybenzene, dicarboxynaphthalene and carboxyanthracene.

Among these, organic acids are preferable, aliphatic carboxylic acids are more preferable, and oxalic acid and citric acid are particularly preferable.

When an acid is added to the solvent, the amount of an acid is preferably in the range of 0.1 to 10 moles per mole of silver atoms in the silver compound is preferable. When two or more kinds of silver compounds are used, the amount of an acid may be set to a range of 0.1 to 10 moles per mole of total silver atoms contained therein.

Examples of the solvent for the carrier dispersion preparation include the same solvents as those mentioned as the solvent for the silver compound solution preparation. As to the solvent for the carrier dispersion preparation and the solvent for the silver compound solution preparation, each solvent is preferably selected as to be miscible to each other. When water is used as the solvent for silver compound solution preparation, water is preferable also as the solvent for carrier dispersion preparation.

In addition, an acid or a base may be added to the solvent for carrier dispersion preparation.

As the acid, the same acids as those mentioned as acids that may be optionally added to the solvent for silver compound solution preparation may be used.

As the base, nitrogen-containing compounds having alkalinity, e.g., amine compounds, imine compounds, hydrazine or hydrazine compounds, ammonia, hydroxylamine, hydroxyamine and ammonium hydroxide are usable, and in addition to nitrogen-containing compounds, alkaline metal hydroxides and the like may also be used.

For these acids and bases, those appropriate or optimal may be selected according to the kinds of the carrier and solvent, or the like.

Although the mixing method of the silver compound solution and the carrier dispersion is not particularly limited, preferred is a method which comprises mixing them while adding one of the two in small amounts to the other. More preferred is a method which comprises dropping a silver compound solution to a carrier dispersion is more preferable.

The temperature when mixing the silver compound solution and the carrier dispersion is selected from the range of 0 to 100° C., and it is preferable to adjust the dropping rate while maintaining this temperature range when the silver compound solution is dropped to the carrier dispersion. After completion of dropping, it is preferable to stir for approximately 0.1 to 10 hours to mix the silver compound and the carrier sufficiently.

The proportion between the silver compound and the carrier used is determined so that the content of silver in the silver catalyst is in the preferred range described above. The amount of the carrier used is preferably 0.1 to 200 parts by mass per part by mass of silver atoms in the silver compound.

The composition obtained from the above-mentioned Step 1 (hereinafter, this composition is called “composition I”) is contained in the solvents used in the preparation of the silver compound solution and the carrier dispersion. The composition I may be subjected to the reduction treatment without collecting it from the solvent after mixing the silver compound solution and the carrier dispersion. The composition I may be collected from the mixed solution obtained by the above-mentioned mixing by a well-known method such as filtration. The composition I is preferably sufficiently washed with a solvent such as water when it is collected from the above-mentioned mixed solution. The amount of alkaline metal component incorporated into the composition I can be decreased by such washing. Since the performance of the silver catalyst thus obtained (catalytic activity) tends to decline from incorporation of an excessive amount of the alkaline metal component, it is preferable to sufficiently remove the alkaline metal component by performing such washing. In addition, if using the same solvent as the solvent used in silver compound solution preparation is used for the washing, the silver compound adhering to the composition I in a trace amount can be sufficiently removed.

The composition I may be subjected to reduction treatment in a state in which the composition I is wetted with the solvent used in filtration, washing and the like, or to reduction treatment after drying by heating, reducing pressure or drying treatment combining these.

Next, reduction treatment in Step 2 will be explained.

This reduction treatment is for converting the silver compound contained in the composition I, specifically silver ions contained in the silver compound, to metallic silver, and more specifically is for converting the silver ions (monovalent or divalent silver ions) to silver atoms of zero valence. The reduction treatment preferably converts substantially all silver compounds contained in the composition I to metallic silver.

When the composition I is subjected to reduction treatment in the mixed solution obtained by mixing the silver compound solution and the carrier dispersion, the reduction treatment may be performed by adding, to the composition I, a reducing agent such as an alcohol such as methanol, ethanol, propanol, butanol, ethylene glycol, propylene glycol, glycerin, aminoethanol or dimethylaminoethanol; a sugar such as glucose, fructose or galactose; an aldehyde such as formaldehyde, acetaldehyde, propionaldehyde, butyraldehyde or phenylaldehyde; a hydrazine such as hydrazine, methylhydrazine, ethylhydrazine, propylhydrazine, butylhydrazine or phenylhydrazine; a metal hydride such as lithium hydride, sodium hydride, potassium hydride, calcium hydride or magnesium hydride; a boron compound such as boron hydride, sodium borohydride, potassium borohydride or dimethylamineborane; and a phosphoric acid such as sodium hypophosphite or potassium hypophosphite.

The amount of the reducing agent may be adjusted based on the amount of a silver compound used in the composition I preparation, and it is preferably at least 1 mole per mole of silver atoms in the silver compound. The conditions relating to the reduction treatment may be appropriately adjusted according to the kind of silver compound or reducing agent and the like.

When the composition I is subjected to reduction treatment in a solvent, the reducing gas described later may be used. In this case, reducing gas may be bubbled into the mixed solution of the composition I may be used, or the composition I may be enclosed in a suitable pressure tight chamber, and the reducing gas introduced therein.

When the composition I is collected in the form of a solid, the reduction treatment can be performed in reducing gas.

When the composition I is subjected to the reduction treatment in reducing gas, for example, the reduction treatment can be performed simply by filling a suitable packed tube with the composition I, and flowing reducing gas into the packed tube. When reducing gas is flowed into a packed tube, the packed tube may be filled with the composition I molded into a suitable shape in order to make the flow characteristics of the reducing gas favorable. Molding of the composition I can be performed by a well-known method such as spray drying and extrusion molding.

Examples of the reducing gas include hydrogen, carbon monoxide, methane, ethane, propane, butane, ethylene, propylene, butane, butadiene and a mixed gas of at least two kinds selected from these. Among these, carbon monoxide, hydrogen and propylene are preferred. In addition, the reducing gas may be diluted, for example, with nitrogen, helium, argon, steam or a dilution gas in which at least two kinds selected from these are mixed, and the mixing ratio thereof is arbitrary.

When the dilution gas is used, the dilution gas may be made to be accompanying when flowing the reducing gas into the packed tube. To give a preferred example, the mixing ratio of steam in the mixed gas flowing inside the packed tube is preferably 5 to 70% by volume when hydrogen is used as the reducing gas and steam as the dilution gas.

For the treatment temperature of the reduction treatment, the optimum temperature may be selected from the range of 20 to 300° C. according to the kind of reducing gas and composition of the composition I, or the kinds of the reducing gas or the dilution gas. However, if the treatment temperature is excessively high, agglomeration of metallic silver particles may occur easily by the reduction treatment, and the effective surface area in the silver catalyst may decrease. As a result, the upper limit for the treatment temperature is preferably not more than 250° C., and more preferably not more than 220° C.

The silver-containing composition is prepared by the reduction treatment.

Step 3 is a step of bringing the silver-containing composition obtained in Step 2 and (b) into contact with each other. The contact of this silver-containing composition and (b) is preferably performed in the presence of a solvent. Examples of such a solvent include those mentioned as a solvent used for the preparation of the silver compound solution or the carrier dispersion, and the optimum solvent may be selected according to the silver-containing composition and the kind of (b).

The amount of (b) used in Step 3 may be adjusted based on the content of silver in the silver-containing composition. More specifically, a range of 0.005 to 10 moles of (b) per mole of silver (atom basis) is preferable, and a range of 0.01 to 1 mole is more preferable.

Preferred operations in Step 3 will be explained. First, using a phosphorus-containing compound as (b), a phosphorus-containing compound solution is prepared by dissolving the phosphorus containing-compound in a suitable solvent. Since the solubility of the phosphorus-containing compound is satisfactory, the solvent used in phosphorus-containing compound solution preparation is preferably water. Further, the phosphorus-containing compound solution is supplemented with a silver-containing composition, and stirred for a predetermined time.

Alternatively, the silver-containing composition and (b) may be mixed without preparing a solution of (b), and these may be washed with a solvent as necessary. When red phosphorus is used as (b), pulverizing and mixing of the silver-containing composition and red phosphorus can be performed using a mortar or the like.

The silver catalyst is prepared by sufficiently bringing the silver-containing composition and phosphorus-containing compound into contact with each other by such a simple operation.

For the temperature when contacting and mixing the silver-containing composition and the phosphorus-containing compound, the temperature is preferably in the range of 0 to 100° C., and more preferably in the range of 20 to 80° C. When the temperature is in this range, a mixing time of about 0.5 to 8 hours is sufficient. Such a preferred temperature can also be appropriately optimized depending on the kind of solvent used in the phosphorus-containing compound solution preparation, when a phosphorus-containing compound is used after making into a phosphorus-containing compound solution.

When a phosphorus-containing compound solution is used, a silver catalyst can be obtained by removing the solvent used in preparation after having sufficiently brought the silver-containing composition and the phosphorus-containing compound solution into contact with each other. Filtration or vacuum distillation may be employed as the removal method of this solvent.

Furthermore, molding may also be subsequently performed after Step 3. Molding may be performed by a well-known method such as spray drying and extrusion molding. Molding may be performed after Step 1 or Step 2.

The silver catalyst thus obtained is used in the method for producing propylene oxide of the present invention without alteration or after having performed drying or the like as necessary.

### <Method for Producing Propylene Oxide>

Next, the method for producing propylene oxide of the present invention (hereinafter referred to as “present production method”) will be explained. The present production method is a method of reacting propylene and oxygen to produce propylene oxide by bringing propylene, oxide and the silver catalyst as described above into contact with each other in the presence of water. Hereinafter, the reaction between oxygen and propylene of the present production method is also referred to as “present reaction”.

The present production method may be performed in a reaction vessel of either batch type or continuous type. From an industrial point of view, it is preferably performed in a reaction vessel of continuous type.

In the present production method, the amount of silver catalyst is preferably at least 0.00005 moles and more preferably at least 0.0001 moles in terms of metallic silver, per mole of propylene. The upper limit thereof is not particularly limited, and a larger amount of propylene oxide can be produced if increasing the amount of silver catalyst. However, the upper limit of the amount of the silver catalyst is adjusted by taking into consideration economic efficiency such as the cost of silver catalyst. Usually, the amount of silver catalyst is not more than 1 mole in terms of metallic silver per mole of propylene.

In the present production method, as water, steam may be used and the silver catalyst may be used by wetting with water. When steam is used as water, a mixed gas obtained by mixing water, oxygen and propylene may be brought into contact with the silver catalyst. It is preferable to use water as steam.

The amount of water is preferably in the range of about 0.1 to about 20 moles per mole of propylene, more preferably in the range of 0.2 to 10 moles, and still more preferably in the range of 0.3 to 8 moles. The above-mentioned “amount of water” indicates an amount of water supplied separately from water contained in air when air is supplied as oxygen.

Oxygen used in the present production method may be only oxygen, i.e. high-purity oxygen, or may be a mixed gas of a gas inert in the present reaction, e.g., nitrogen, carbon dioxide, and the like) and oxygen, e.g., air. The amount of oxygen can be appropriately adjusted according to the reaction mode (continuous type or batch type), kind of silver catalyst and the like. The amount of oxygen is preferably in the range of 0.01 to 100 moles per mole of propylene, and more preferably in the range of 0.03 to 30 moles. The reaction temperature is preferably in the range of 100 to 400° C., and more preferably in the range of 120 to 300° C.

Furthermore, in the present production method, propylene oxide can be produced in a higher yield by performing the present production method in the presence of a halogen compound, particularly an organic halogenated compound. Examples of the halogen compound include the halogen compounds disclosed in Japanese Unexamined Patent Application Publication No. 2008-184456, and it is preferably a organic chlorinated compound. Examples of the organic chlorinated compound include chloroethane, 1,2-dichloroethane, chloromethane and vinyl chloride. The halogen compound is preferably a compound existing in the form of a gas at the temperature and pressure condition in the reaction system of the present reaction.

The amount of the halogen compound is preferably 1 to 1000 ppm by volume, and more preferably 1 to 500 ppm by volume based on a total volume of the mixed gas other than steam, i.e. a mixed gas composed of oxygen and propylene and a dilution gas added as necessary.

The reaction pressure of the present production method is not limited, and may be selected from those in reduced pressure conditions to pressurized conditions. The pressure under pressurized conditions is preferable in the point of allowing sufficient contact of oxygen and propylene with the silver catalyst, it may be a reaction pressure selected from the range of 0.01 to 3 MPa in absolute pressure, and is more preferably selected from the range of 0.02 to 2 MPa. The reaction pressure is determined by also taking into consideration the pressure resistibility of the reaction device used in the present production method. The reduced pressure condition means a pressure lower than the atmospheric pressure. The pressurized condition means a pressure higher than the atmospheric pressure.

Hereinafter, an embodiment of the present production method of a continuous type, which is a favored reaction mode, will be explained.

First, the silver catalyst in a predetermined amount is filled into a reaction tower equipped with a gas supply port and a gas exhaust port. Suitable heating means may be provided in the reaction tower, and the inside of the reaction tower may be raised in temperature up to a predetermined reaction temperature by such heating means. Subsequently, using a compressor or the like, a source gas containing propylene, oxygen and steam is supplied from the gas supply port into the reaction tower. The halogen compound may be contained in this source gas as described above. This source gas contacts with the silver catalyst inside the reaction tower so that propylene contacts with oxygen in the presence of the silver catalyst and water. Propylene oxide is generated by the reaction of propylene and oxygen contained in the gas, and the product gas containing the propylene oxide thus generated is exhausted from the gas exhaust port.

The linear velocity of the source gas that is passed through the inside of a reaction tower is determined so as to make a residence time that allows the source gas and the present silver catalyst to sufficiently generate propylene oxide.

Although a case of heating means being provided in the reaction tower has been described in the above embodiment, it may be a mode in which the reaction tower may be maintained at ambient temperature, and the source gas may be supplied and heated up to a predetermined reaction temperature by appropriate heating means, and then supplied into the reaction tower. It may be a mode in which suitable stirring means is provided in the reaction tower, and a source gas is supplied while stirring the silver catalyst that is present inside the reaction tower.

The propylene oxide thus generated, unreacted propylene and oxygen, and byproducts such as carbon dioxide may be contained in the product gas passing through the reaction tower. In addition, in a case of using propylene and oxygen after dilution, an inert gas used for dilution may be incorporated. After having collected this product gas, propylene oxide, which is the objective, can be removed by separation means such as distillation.

### EXAMPLES

Hereinafter, the present invention will be explained in further detail by way of Examples.

### Preparation Example 1

Mixed were 7.5 g of strontium carbonate (Sakai Chemical Industry, Co. Ltd., trade name SW-K) and 50 g of water, and the temperature of the mixture thus obtained was set to 20 to 25° C. The same temperature was maintained, and after 1.4 g of sodium hydroxide (Nacalai Tesque, Inc., 99% purity) was added to the mixture, the mixed solution thus obtained was stirred for several minutes while cooling using an ice bath, thereby preparing a carrier dispersion. 13.96 g of a silver nitrate aqueous solution (silver compound solution) containing 3.96 g of silver nitrate (Nacalai Tesque, Inc., 99% purity) was dropped into this carrier dispersion, and the mixed solution thus obtained was stirred for further 3 hours. Thereafter, a composition in the form of a solid was obtained by filtering the mixed solution, and washing the solid thus obtained with about 800 mL of ion-exchanged water. Next, a glass tube for calcination (inner diameter of 2 cm) was filled with the composition in a solid form, the temperature was raised over 1.5 hours up to 110° C. while flowing each gas under the conditions of 5 mL/min of CO (carbon monoxide) gas, 50 mL/min of nitrogen gas and 1 mL/hour of water (=steam), then further raising the temperature up to 150° C., maintaining the same temperature for 1 hour, further raising the temperature over 5 hours to 210° C., followed by cooling, thereby performing reduction treatment to obtain the silver-containing composition A.

Furthermore, a phosphoric acid aqueous solution (5.168 g) containing 0.168 g of phosphoric acid was dropped to 3 g of silver-containing composition A, and the suspension thus obtained was stirred for 1 hour under a condition of 20° C. Subsequent to completion of stirring, the aqueous component was removed using a rotary evaporator, thereby obtaining catalyst 1.

### Preparation Example 2

An aqueous phosphorous acid solution (5.119 g) containing 0.119 g of phosphorous acid was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 2.

### Preparation Example 3

An aqueous hypophosphorous acid solution (5.192 g) containing 0.192 g of hypophosphorous acid was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 3.

### Preparation Example 4

An aqueous ammonium dihydrogen phosphate solution (5.042 g) containing 0.042 g of ammonium dihydrogen phosphate was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 4.

### Preparation Example 5

An aqueous ammonium dihydrogen phosphate solution (5.190 g) containing 0.190 g of ammonium dihydrogen phosphate was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 5.

### Preparation Example 6

An aqueous ammonium dihydrogen phosphate solution (5.481 g) containing 0.481 g of ammonium dihydrogen phosphate was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 6.

### Preparation Example 7

An aqueous diammonium hydrogen phosphate solution (5.048 g) containing 0.048 g of diammonium hydrogen phosphate was dropped to 3 g of silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 7.

### Preparation Example 8

An aqueous ammonium phosphate solution (5.074 g) containing 0.074 g of ammonium phosphate trihydrate was dropped to 3 g of a silver-containing composition A obtained in the same manner as Preparation Example 1, and the suspension thus obtained was stirred for 1 hour under a condition of 20° C. Subsequent to completion of stirring, the aqueous component was removed using a rotary evaporator, thereby obtaining catalyst 8.

### Preparation Example 9

Experimentation in the same manner as Preparation Example 1 was performed, except that hydrogen gas was supplied at 5 mL/min and nitrogen gas was supplied at 50 mL/min instead of supplying CO (carbon monoxide) gas at 5 mL/min, nitrogen gas at 50 mL/min and steam at 1 mL/hour, thereby obtaining a silver-containing composition B.

An aqueous ammonium dihydrogen phosphate solution (5.014 g) containing 0.014 g of ammonium dihydrogen phosphate was dropped to 1.5 g of the silver-containing composition B, and the suspension thus obtained was stirred for 1 hour under a condition of 20° C. Subsequent to completion of stirring, the aqueous component was removed using a rotary evaporator, thereby obtaining catalyst 9.

### Preparation Example 10

A catalyst for comparison was prepared by the method disclosed in Japanese Examined Patent Application Publication No. S51-40051, Example 5. A precipitate of silver oxide was generated by dissolving 23.7 g of silver nitrate (Wako Pure Chemical Industries, Ltd., 99.8% purity) in 300 mL of deionized water, and dropping a potassium hydroxide aqueous solution (342.9 g) containing 42.9 g of potassium hydroxide (Wako Pure Chemical Industries, Ltd., 85% purity) onto this. 10.9 g of a 37% formalin solution (Wako Pure Chemical Industries, Ltd.) was dropped into the mixed solution thus obtained, after leaving to stand for 30 minutes, it was heated at 60° C. and the precipitate thus generated was suction filtered and washed with deionized water. It was further treated with 500 mL of a 2% by mass nitric acid aqueous solution at a temperature of 20° C. for 30 minutes, filtered and washed, followed by drying to obtain active silver powder. Among the active silver powder obtained in this way, that from 32 mesh to 100 mesh was sifted out, 4 mL of this was immersed in 100 g of an aqueous solution containing 1.09 mg of hexamethylphosphortriamide, and then evaporated to dryness to obtain a catalyst for comparison.

### Comparative Example 1

1 mL of the silver-containing composition A obtained in Preparation Example 1 was filled into a half-inch stainless-steel reaction tube, and each gas was supplied to the reaction tube under the conditions of 450 mL/hour of propylene gas, 900 mL/hour of air, 990 mL/hour of nitrogen gas and 1.2 mL/hour of water (=steam) at 0.4 MPa gauge pressure and a reaction temperature of 200° C. The composition of the product gas emitted from the reaction tube was analyzed by gas chromatography.

### Example 1

Except that catalyst 1 was used instead of the silver-containing composition A, experimentation was performed in the same manner as Comparative Example 1.

### Example 2

Except that catalyst 2 was used instead of the silver-containing composition A, experimentation was performed in the same manner as Comparative Example 1.

### Example 3

Except that catalyst 3 was used instead of the silver-containing composition A, experimentation was performed in the same manner as Comparative Example 1.

TABLE 1

| | Comparative | | | |
|---|---|---|---|---|
| | Example 1 | Example 1 | Example 2 | Example 3 |
| Silver | Silver-containing | Catalyst 1 | Catalyst 2 | Catalyst 3 |
| Catalyst | composition A | | | |
| Propylene | 7 | 28 | 39 | 32 |
| Oxide | | | | |
| Selectivity | | | | |
| (%) | | | | |
| Propylene | 156 | 104 | 29 | 27 |
| Oxide Yield | | | | |
| (μmol · PO/Hr) | | | | |

### Example 4

Except that catalyst 4 was used instead of the silver-containing composition A, experimentation was performed in the same manner as Comparative Example 1.

### Example 5

Except that catalyst 5 was used instead of the silver-containing composition A, experimentation was performed in the same manner as Comparative Example 1.

### Example 6

Except that catalyst 6 was used instead of the silver-containing composition A, experimentation was performed in the same manner as Comparative Example 1.

### Example 7

Except that catalyst 7 was used instead of the silver-containing composition A, experimentation was performed in the same manner as Comparative Example 1.

### Example 8

Except that catalyst 8 was used instead of the silver-containing composition A, experimentation was performed in the same manner as Comparative Example 1.

TABLE 2

| | Example 4 | Example 5 | Example 6 | Example 7 | Example 8 |
|---|---|---|---|---|---|
| Silver | Catalyst 4 | Catalyst 5 | Catalyst 6 | Catalyst 7 | Catalyst 8 |
| Catalyst | | | | | |
| Propylene | 31 | 29 | 25 | 25 | 40 |
| Oxide | | | | | |
| selectivity | | | | | |
| (%) | | | | | |
| Propylene | 88 | 90 | 55 | 102 | 64 |
| Oxide Yield | | | | | |
| (μmol · | | | | | |
| PO/Hr) | | | | | |

### Comparative Example 2

Except that the silver-containing composition B was used instead of the silver-containing composition A, experimentation was performed in the same manner as Comparative Example 1.

### Example 9

Except that catalyst 9 was used instead of the silver-containing composition A, experimentation was performed in the same manner as Comparative Example 1.

TABLE 3

| | Comparative | |
|---|---|---|
| | Example 2 | Example 9 |
| Silver Catalyst | Silver-containing | Catalyst 9 |
| | composition B | |
| Propylene Oxide | 5 | 28 |
| Selectivity (%) | | |
| Propylene Oxide Yield | 124 | 104 |
| (μmol · PO/Hr) | | |

### Comparative Example 3

Except that the catalyst for comparison was used instead of the silver-containing composition A, experimentation was performed in the same manner as Comparative Example 1. The reaction results thereof are shown in Table 4.

TABLE 4

| | Comparative Example 3 |
|---|---|
| Silver Catalyst | Catalyst for comparison |
| Propylene Oxide Selectivity (%) | 17 |
| Propylene Oxide Yield | 2 |
| (μmol · PO/Hr) | |

### INDUSTRIAL APPLICABILITY

According to the present invention, propylene oxide can be produced in high yield from propylene and oxygen. Propylene oxide is extremely useful as a raw material of various manufactured products and the like.

1. A method for producing propylene oxide, comprising a step of bringing propylene, oxygen and a silver catalyst into contact with each other in the presence of water, wherein the silver catalyst is a catalyst prepared from (a) metallic silver, a silver compound or a mixture thereof, (b) phosphorus, a phosphorus-containing compound or a mixture thereof and (c) a carrier.

2. The method according to claim 1, wherein the (c) comprises a metal carbonate excepting silver-containing metal carbonates, a metal oxide excepting silver-containing metal oxides or carbon as a main component.

3. The method according to claim 1, wherein the (c) comprises an alkaline earth metal carbonate.

4. The method according to claim 1, wherein the amount of the (b) contained in the silver catalyst is in the range of 0.001 to 5 moles per mole of silver contained in the (a).

5. The method according to claim 1, wherein the (b) is a phosphorus-containing compound.

6. The method according to claim 1, wherein the silver catalyst is prepared by a method comprising:
a step of bringing the (a) and the (c) into contact with each other to obtain a composition thereof; and
a step of bringing the composition and the (b) into contact with each other.

7. The method according to claim 1, wherein the (a) is a silver compound or a mixture of metallic silver and a silver compound.

8. The method according to claim 7, wherein the silver catalyst is prepared by a method comprising:
a step of bringing the (a) and the (c) into contact with each other to obtain a composition thereof;
a step of reducing the silver compound contained in the composition; and
a step of bringing the composition after reduction and the (b) into contact with each other.

9. The method according to claim 1, wherein the (a) is at least one silver compound selected from the group consisting of silver nitrate, silver carbonate and silver oxide.

10. The method according to claim 1, wherein the silver catalyst contains silver in an amount of at least 0.5% by mass based on the mass of the silver catalyst.

11. The method according to claim 1, wherein the amount of water is in the range of 0.2 to 10 moles per mole of propylene.

12. A silver catalyst for production of olefin oxide which is a catalyst prepared from (a) metallic silver, a silver compound or a mixture thereof, (b) phosphorus, a phosphorus-containing compound or a mixture thereof and (c) a carrier.

13. The silver catalyst according to claim 12, wherein the (a) is a silver compound, said silver catalyst being prepared by bringing the (a) and the (c) into contact with each other to obtain a composition thereof, reducing the silver compound contained in the composition, and then bringing the composition after reduction and the (b) into contact with each other.

14. The silver catalyst according to claim 12, wherein the (c) comprises a metal carbonate excepting silver-containing metal carbonates, a metal oxide excepting silver-containing metal oxides or carbon as a main component.

15. The silver catalyst according to claim 12, wherein the silver compound is selected from the group consisting of silver nitrate, silver carbonate and silver oxide.

16. The silver catalyst according to claim 12, wherein the (c) comprises an alkaline earth metal carbonate.

17. The silver catalyst according to claim 12, wherein the (a) is at least one silver compound selected from the group consisting of silver nitrate, silver carbonate and silver oxide.

18. The silver catalyst according to claim 12, wherein the olefin oxide is propylene oxide.
